(19) United States
(12) Patent Application Publication
(10) Pub. No.: US 2024/0238002 A1
(43) Pub. Date: Jul. 18, 2024

(54) THROMBUS REMOVAL SYSTEMS AND ASSOCIATED METHODS

(71) Applicant: Shifamed Holdings, LLC, Campbell, CA (US)

(72) Inventors: Aadel AL-JADDA, San Carlos, CA (US); Tom SAUL, Portland, OR (US); Kevin MULLER, San Carlos, CA (US); Amr SALAHIEH, Saratoga, CA (US)

(21) Appl. No.: 18/562,038

(22) PCT Filed: May 19, 2022
(86) PCT No.: PCT/US2022/030039

§ 371 Date: Nov. 17, 2023

Related U.S. Application Data
(60) Provisional application No. 63/190,784, filed on May 19, 2021.

(51) Int. Cl. A61B 17/3203; A61B 17/00; A61B 17/22

(52) U.S. Cl. CPC A61B 17/32037; A61B 17/00234; A61B 17/22; A61B 2017/00238; A61B 2017/00292; A61B 2017/22079; A61B 2217/005; A61B 2217/007

(57) ABSTRACT

The present technology relates to systems and methods for removing a thrombus from a blood vessel of a patient. In some embodiments, the present technology is directed to systems including an elongated catheter having a distal portion configured to be positioned within the blood vessel of the patient, a proximal portion configured to be external to the patient, and a lumen extending therebetween. The system can also include a fluid delivery mechanism coupled with a fluid lumen and configured to apply fluid to at least partially fragment the thrombus, and an aspiration mechanism fluidly coupled to an aspiration lumen and configured to aspirate the fragmented thrombus.

### RELATED APPLICATIONS

This application is related to International Application No. PCT/US2021/020915, filed Mar. 4, 2021, and U.S. Application No. 63/190,784, filed May 19, 2021, the disclosures of which are incorporated by reference herein.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present technology generally relates to medical devices and, in particular, to systems including aspiration and fluid delivery mechanisms and associated methods for removing a thrombus from a mammalian blood vessel.

### BACKGROUND

Thrombotic material may lead to a blockage in fluid flow within the vasculature of a mammal. Such blockages may occur in varied regions within the body, such as within the pulmonary system, peripheral vasculature, deep vasculature, or brain. Pulmonary embolisms typically arise when a thrombus originating from another part of the body (e.g., a vein in the pelvis or leg) becomes dislodged and travels to the lungs. Anticoagulation therapy is the current standard of care for treating pulmonary embolisms, but may not be effective in some patients. Additionally, conventional devices for removing thrombotic material may not be capable of navigating the vascular anatomy of the lungs, may not be effective in removing thrombotic material, and/or may lack the ability to provide sensor data or other feedback to the clinician during the thrombectomy procedure.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1-1E illustrate various views of a portion of a thrombus removal system including a distal portion of an elongated catheter configured in accordance with an embodiment of the present technology.

FIGS. 2A-2E illustrate plan views of various configurations of irrigation ports and fluid streams of a thrombus removal system according to embodiments of the present technology.

FIGS. 3A-3H illustrate an elevation view of various configurations of irrigation ports of a thrombus removal system according to embodiments of the present technology.

FIGS. 4A-4H illustrate an elevation view of various configurations of irrigation ports and fluid streams of a thrombus removal system according to embodiments of the present technology.

FIGS. 5A-5G illustrate various configurations of irrigation ports of a thrombus removal system according to embodiments of the present technology.

FIG. 6 is a diagram showing a relationship between jet flow rate and interaction with one or more clots.

### SUMMARY OF THE DISCLOSURE

A thrombus removal system is provided, comprising an elongated catheter device including a distal portion configured to be positioned within a blood vessel of a patient, the distal portion comprising an inner wall forming an aspiration lumen, and an outer wall, a manifold formed near a distal end of the fluid lumen, the manifold including at least two fluid ports formed therein and adapted for fluid communication with the fluid lumen to direct respective fluid streams from the at least two fluid ports, a proximal portion configured to be positioned external to the patient, the aspiration lumen extending from the distal portion to the proximal portion, an aspiration mechanism positioned external to the patient and fluidly coupled with the aspiration lumen, the aspiration mechanism configured to reduce a pressure at the distal portion (a) to engage a thrombus therewith and/or (b) to draw the thrombus and/or thrombus fragments proximally, and a fluid delivery mechanism configured to supply fluid through the fluid lumen.

In some embodiments, the inner wall and the outer wall are concentrically arranged such that the fluid lumen is generally annular in cross-section.

In one example, the system further includes at least one fluid wall disposed in the space between the inner and outer walls, the at least one fluid wall forming the fluid lumen.

In some examples, the irrigation manifold is formed from the inner wall and the outer wall.

In one embodiment, the at least two fluid ports are formed within the inner wall of the manifold.

In some embodiments, the inner wall has a first thickness in a first region within the manifold, and a second thickness proximal to the first region. In other examples, the first thickness is greater than the second thickness. In one embodiment, the first thickness is about twice that of the second thickness. In some implementations, the first thickness ranges from 0.10 mm to 0.60 mm and the second thickness ranges from 0.20 mm to 0.70 mm. In some embodiments, the first thickness is selected to provide a generally laminar flow for the respective fluid streams.

In one example, a cross-sectional dimension of the two or more ports varies along its length.

In some implementations, the two or more ports are conical along their lengths.

In one embodiment, a smallest dimension of the two or more ports is positioned at a distal end of the two or more ports.

In some examples, the respective fluid streams are configured to intersect near at least one intersection region.

In one embodiment, at least one intersection region is located proximally with respect to at least one of the at least two fluid ports.

In other examples, at least one intersection region is located distally with respect to at least one of the at least two fluid ports.

In some embodiments, the irrigation manifold is configured to increase a flow rate of the fluid in the fluid lumen. In another embodiment, the irrigation manifold is configured to increase a pressure of the fluid in the fluid lumen.

In some embodiments, the system further includes a funnel positioned at a distal end of the distal portion, the funnel being configured to engage with the thrombus.

In one embodiment, the irrigation manifold is disposed proximally of the manifold. In other embodiments, the irrigation manifold is integrated into the funnel.

A method for removing a thrombus from a blood vessel of a patient is provided, the method comprising introducing a distal portion of an elongate catheter to a thrombus location in a blood vessel, drawing at least a section of the thrombus into the distal portion, and directing fluid toward the thrombus at between 10-15 m/s from at least two different points along respective fluid paths that intersect.

In some embodiments, the drawing is by suction applied via an aspiration lumen of the elongate catheter.

In another embodiment, directing fluid further comprises directing one of the respective fluid paths proximally. In some embodiments, directing fluid further comprises directing one of the respective fluid paths distally. In other embodiments, directing fluid further comprises directing a first fluid path proximally and a second fluid path distally. In another example, directing fluid further comprises directing a first fluid path proximally and a second fluid path orthogonally with respect to an aspiration flow axis.

A method for removing a thrombus from a blood vessel of a patient is also provided, the method comprising introducing a distal portion of an elongate catheter to a thrombus location in a blood vessel, drawing at least a section of the thrombus into the distal portion, delivering fluid from a fluid delivery mechanism into a fluid lumen of the elongate catheter, increasing a flow rate of the fluid with an irrigation manifold disposed at a distal end of the fluid lumen, and directing at least two fluid streams with ports disposed in the irrigation manifold.

In some embodiments, the drawing is by suction applied via an aspiration lumen of the elongate catheter.

In one embodiment, the at least two fluid streams are directed proximally with the ports. In other embodiments, the at least two fluid streams are directed distally with the ports. In some examples, the at least two fluid streams are directed orthogonally relative to a longitudinal axis of the elongate catheter with the ports. In another embodiment, the at least two fluid streams are directed towards an intersection region.

In some embodiments, the flow rate of the at least two fluid streams comprises 10-15 m/s. In another embodiment, the flow rate of the at least two fluid streams comprises 12-15 m/s.

### DETAILED DESCRIPTION

The present technology is generally directed to thrombus removal systems and associated methods. A system configured in accordance with an embodiment of the present technology can include, for example, an elongated catheter having a distal portion configured to be positioned within a blood vessel of the patient, a proximal portion configured to be external to the patient, a fluid delivery mechanism configured to fragment the thrombus with pressurized fluid, an aspiration mechanism configured to aspirate the fragments of the thrombus, and one or more lumens extending at least partially from the proximal portion to the distal portion.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the examples but are not described in detail with respect to the figures.

Reference throughout this specification to “one embodiment” or “an embodiment” means that a particular feature, structure, or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

Reference throughout this specification to relative terms such as, for example, “generally,” “approximately,” and “about” are used herein to mean the stated value plus or minus 10%.

Although some embodiments herein are described in terms of thrombus removal, it will be appreciated that the present technology can be used and/or modified to remove other types of emboli that may occlude a blood vessel, such as fat, tissue, or a foreign substance. Additionally, although some embodiments herein are described in the context of thrombus removal from a pulmonary artery (e.g., pulmonary embolectomy), the technology may be applied to removal of thrombi and/or emboli from other portions of the vasculature (e.g., in neurovascular, coronary, or peripheral applications). Moreover, although some embodiments are discussed in terms of maceration of a thrombus with a fluid, the present technology can be adapted for use with other techniques for breaking up a thrombus into smaller fragments or particles (e.g., ultrasonic, mechanical, enzymatic, etc.).

The headings provided herein are for convenience only and do not interpret the scope or meaning of the claimed present technology.

### Systems for Thrombus Removal

As provided above, the present technology is generally directed to thrombus removal systems. Such systems include an elongated catheter having a distal portion positionable within a blood vessel of the patient (e.g., an artery or vein), a proximal portion positionable outside the patient's body, a fluid delivery mechanism configured to fragment the thrombus with pressurized fluid, an aspiration mechanism configured to aspirate the fragments of the thrombus, and one or more lumens extending at least partially from the proximal portion to the distal portion. In some embodiments, the systems herein are configured to engage a thrombus in a patient's blood vessel, break the thrombus into small fragments, and aspirate the fragments out of the patient's body. The pressurized fluid streams (e.g., jets) function to cut or macerate thrombus, before, during, and/or after at least a portion of the thrombus has entered the aspiration lumen or a funnel of the system. Fragmentation helps to prevent clogging of the aspiration lumen and allows the thrombus removal system to macerate large, firm clots that otherwise could not be aspirated. As used herein, “thrombus” and “embolism” are used somewhat interchangeably in various respects. It should be appreciated that while the description may refer to removal of “thrombus,” this should be understood to encompass removal of thrombus fragments and other emboli as provided herein.

According to embodiments of the present technology, a fluid delivery mechanism can provide a plurality of fluid streams (e.g., jets) to fluid apertures of the thrombus removal system for macerating, cutting, fragmenting, pulverizing and/or urging thrombus to be removed from a proximal portion of the thrombus removal system. The thrombus removal system can include an aspiration lumen extending at least partially from the proximal portion to the distal portion of the thrombus removal system that is adapted for fluid communication with an aspiration pump (e.g., vacuum source). In operation, the aspiration pump may generate a volume of lower pressure within the aspiration lumen near the proximal portion of the thrombus removal system, urging aspiration of thrombus from the distal portion.

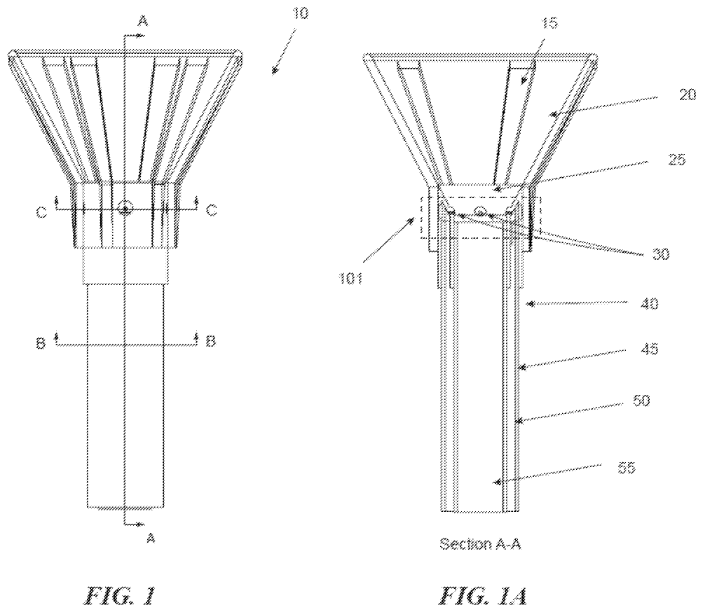

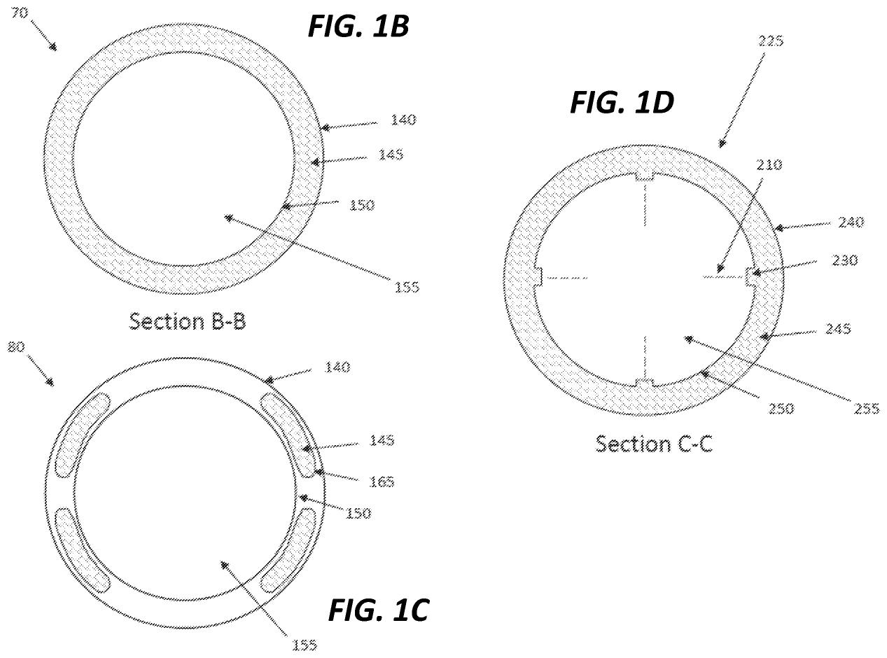

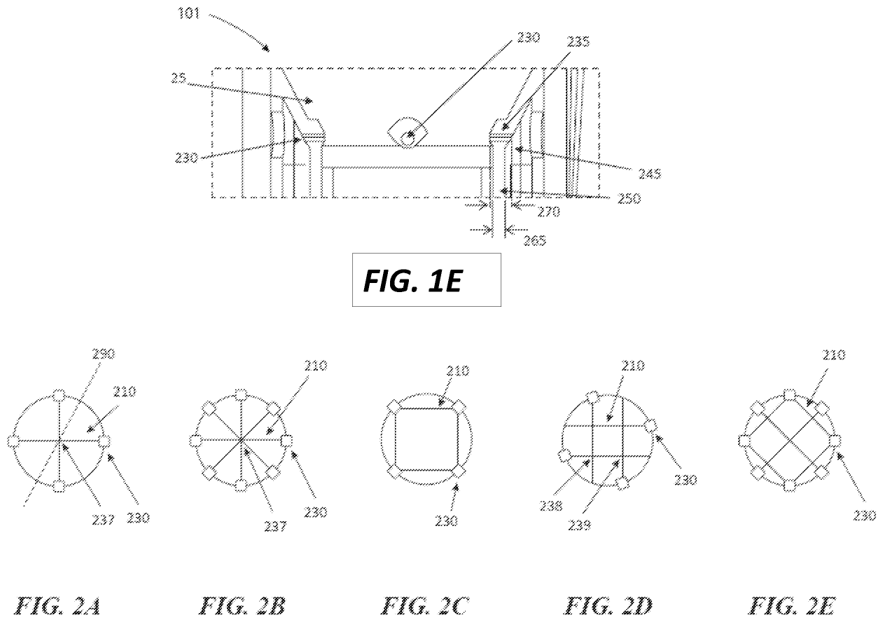

FIG. 1 illustrates a distal portion 10 of a thrombus removal system according to an embodiment of the present technology. FIG. 1 includes cross-sectional lines A-A, B-B, and C-C, which are illustrated in FIG. 1A, FIG. 1B/1C, and FIG. 1D, respectively. FIG. 1A Section A-A illustrates an elevation sectional view of the distal portion. The example section A-A in FIG. 1A depicts a funnel 20 that is positioned at the distal end of the distal portion 10, the funnel adapted to engage with thrombus and/or a tissue (e.g., vessel) wall to aid in thrombus fragmentation and/or removal. The example section A-A in FIG. 1A depicts a double walled thrombus removal device construction having an outer wall/tube 40 and an inner wall/tube 50. An aspiration lumen 55 is formed by the inner wall 50 and is centrally located. A generally annular volume forms at least one fluid lumen 45 between the outer wall 40 and the inner wall 50. The fluid lumen 45 is adapted for fluid communication with a fluid delivery mechanism. One or more apertures (e.g., nozzles, orifices, or ports) 30 are positioned in the thrombus removal system to be in fluid communication with the fluid lumen 45 and an irrigation manifold 25. In operation, the ports 30 are adapted to direct (e.g., pressurized) fluid toward thrombus that is engaged with the distal portion 10 of the thrombus removal system. In some embodiments, the manifold can be positioned proximal to the funnel. In other embodiments, the manifold can be integrated into the funnel.

The thrombus removal system can be sized and configured to access and remove thrombi in various locations or vessels within a patient's body. It should be understood that while the dimensions of the system may vary depending on the target location, generally the same features and components described herein will be implemented in the thrombus removal system regardless of the application. For example, a thrombus removal system configured to remove pulmonary embolism (PE) from a patient may have an outer wall/tube with a size of approximately 11-13 Fr, or preferably 12 Fr, and an inner wall/tube with a size of 7-9 Fr, or preferably 8 Fr. A deep vein thrombosis (DVT) device, on the other hand, may have an outer wall/tube with a size of approximately 9-11 Fr, or preferably 10 Fr, and an inner wall/tube with a size of 6-9 Fr, or preferably 7.5 Fr. Applications are further provided for ischemic stroke and peripheral embolism applications.

Section B-B of FIG. 1B illustrates in plan view a portion of the thrombus removal system that is proximal to the funnel and irrigation manifold. Section B-B depicts an outer wall 140, an inner wall 150, an aspiration lumen 155 and a fluid lumen 145. As shown, the inner wall 150 forms the aspiration lumen, and the inner wall and outer wall are concentric to another. The fluid lumen 145 is formed in the space between the inner wall and outer wall. In some embodiments, in cross-section the aspiration lumen 155 is generally circular and the fluid lumen 145 is generally annular in shape (e.g., cross-section 70). It will be appreciated that alternative constructions and/or arrangements of the inner wall 150 and the outer wall 140 produce variations in cross-sectional shape of the aspiration and fluid lumens 155 and 145. For example, the inner wall 150 can be shaped to form an aspiration lumen 155 that, in cross-section, is generally oval, circular, rectilinear, square, pentagonal, or hexagonal. The inner and outer walls 150 and 140 can be shaped and arranged to form a fluid lumen 145 that, in cross-section, is generally crescent-shaped, diamond shaped, or irregularly shaped. For example, referring to FIG. 1C Section B-B, the region between the inner wall 150 and the outer wall 140 can include one or more wall structures 165 that form respective fluid lumens 145 (e.g., as in cross-section 80). The wall structures 165 can be formed by lamination between the outer and inner walls 140 and 150, or by a multi-lumen extrusion that forms a plurality of the wall structures.

Section C-C of FIG. 1D illustrates in plan view a portion of the thrombus removal system comprising an irrigation manifold 225. Section C-C depicts an outer wall 240, an inner wall 250, a fluid lumen 245, an aspiration lumen 255, and ports 230 for directing respective fluid streams 210.

Detail View 101 of FIG. 1E illustrates a section view in elevation of a portion of the irrigation manifold 25 that includes a plurality of ports 230 that are formed within an inner wall 250. As shown, the manifold can be positioned at or near a distal end of the fluid lumen(s) formed by the inner and outer walls. In some embodiments, a thickness of one or more walls of the thrombus removal system may be varied along its axial length and/or its circumference. As shown in Detail View 101, inner wall 250 has a first thickness 265 in a region 250 that is proximal to the irrigation manifold 25, and a second thickness 270 in a region 235 that includes the ports 230. In some embodiments, the second thickness 270 is greater than the first thickness 265. The first thickness 265 can correspond to a general wall thickness of the inner wall 50 and/or of the outer wall 40, which can be from about 0.10 mm to about 0.60 mm, or any value within the aforementioned range. The second thickness 270 can be from about 0.20 mm to about 0.70 mm, from about 0.70 mm to about 0.90 mm, or from about 0.90 mm to about 1.20 mm. The second thickness 270 can be any value within the aforementioned range. The dimension of the second thickness 270 can be selected to provide a fluid path through the ports 230 that produces a generally laminar flow for a fluid stream that is directed therethrough, when the fluid delivery mechanism supplies fluid via the fluid lumen 245 at a typical operating pressure. Such operating pressure can be from about 10 psi to about 60 psi, from about 60 psi to about 100 psi, or from about 100 psi to about 150 psi. The operating pressure of the fluid delivery mechanism can be any value within the aforementioned range of values. In some embodiments, the fluid delivery mechanism is operated in a high pressure mode, having a pressure from about 150 psi to about 250 psi, from about 250 psi to about 350 psi, from about 350 psi to about 425 psi, or from about 425 psi to about 500 psi. The operating pressure of the fluid delivery mechanism in the high pressure mode can be any value within the aforementioned range of values.

The manifold is configured to increase a fluid pressure and/or flow rate of the fluid. When fluid is provided by the fluid delivery mechanism to the fluid lumen(s) at a first pressure and/or a first flow rate, the manifold is configured to increase the pressure of the fluid to a second pressure and/or is configured to increase the flow rate of the fluid to a second flow rate. The second pressure and/or second fluid rate can be higher than the first pressure and/or first flow rate. As a result, the manifold can be configured to increase the relatively low operating pressures and/or flow rates generated by the fluid delivery mechanism to the relatively high pressures and/or high flow rates generated by the ports/fluid streams.

In some embodiments, a profile (cross-sectional dimension) of a port 230 varies along its length (e.g., is non-cylindrical). A variation in the cross-sectional dimension of the port may alter and/or adjust a characteristic of fluid flow along the port 230. For example, a reduction in cross-sectional dimension may accelerate a flow of fluid through the port 230 (for a given volume of fluid). In some embodiments, a port 230 may be conical along its length (e.g., tapered), such that its smallest dimension is positioned at the distal end of the port 230, where distal is with respect to a direction of fluid flow.

In some embodiments, the port 230 is formed to direct the fluid flow along a selected path. FIGS. 2A-2E illustrate various embodiments of arrangements of ports 230 for directing respective fluid streams 210. In some embodiments, such as those shown in FIGS. 2A and 2B, at least two ports 230 are arranged to produce (e.g., respective) fluid streams 210 that intersect at an intersection region 237 of the thrombus removal system. An intersection region 237 can be a region of increased fluid momentum and/or energy transfer, which increase is with respect to individual fluid streams that are not directed to combine at the intersection. The increased fluid momentum and/or energy transfer at an intersection may advantageously fragment thrombus more efficiently and/or quickly. In some embodiments, an intersection region can be formed from at least 2, at least 3, at least 4, at least 5, at least 6, at least 7, at least 8, at least 9, or at least 10 fluid streams 210. An intersection region can be generally near a central axis 290 of the thrombus removal system (e.g., 237), or away from the central axis (e.g., 238 and 239 in the embodiment of FIG. 2D). In some embodiments, at least two intersection regions (e.g., 238 and 239) are formed. In some embodiments, one or more ports 230 are arranged to direct a fluid stream 210 along an oblique angle with respect to the central axis of the thrombus removal system. An operating pressure of the fluid delivery mechanism may be selected to approach a targeted fluid velocity for a fluid stream 210 that is delivered from a port 230. The targeted fluid velocity for a fluid stream 210 can be about 5 meters/second (m/s), about 8 m/s, about 10 m/s, about 12 m/s, about 10-15 m/s, or about 12-15 m/s. The targeted fluid velocity for fluid stream 210 can be any value within the range of aforementioned values. In some embodiments, at least two ports 230 are adapted to delivery respective fluid streams at different fluid velocities, for a given pressure of the fluid delivery mechanism. In some embodiments, at least two ports 230 are adapted to deliver respective fluid streams at the substantially the same fluid velocities, for a given pressure of the fluid delivery mechanism. In some embodiments, angular momentum is imparted to a thrombus by application of a) at least one fluid stream 210 that is directed at an oblique angle from a port 230, and/or b) at least two fluid streams 210 that have different fluid velocities. Advantageously, angular momentum produced in a thrombus may impart a (e.g., centrifugal) force that assists in fragmentation and removal of the thrombus. Advantageously, an increased cross-sectional area of the fluid lumen 145 reduces a required operating pressure of the fluid delivery mechanism to achieve a targeted fluid velocity of the fluid streams.

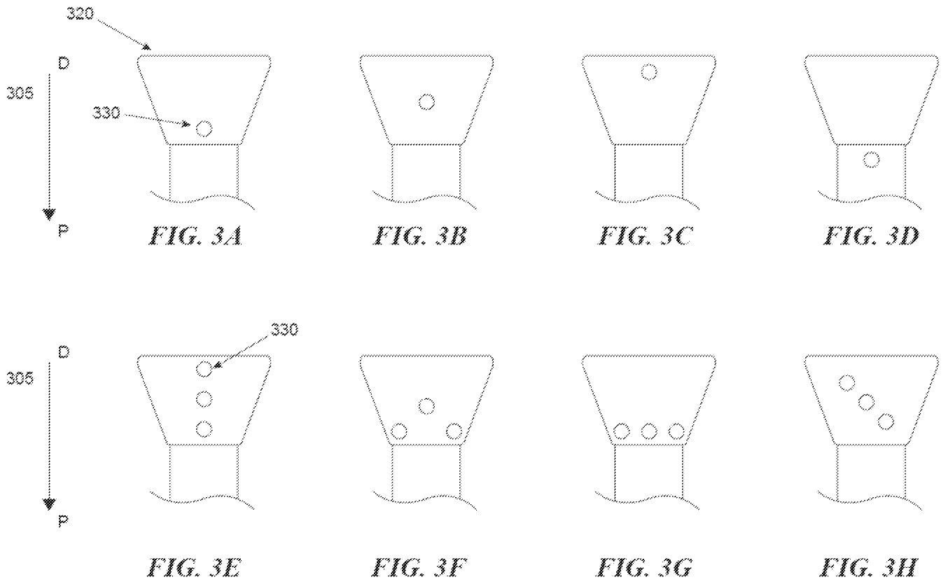

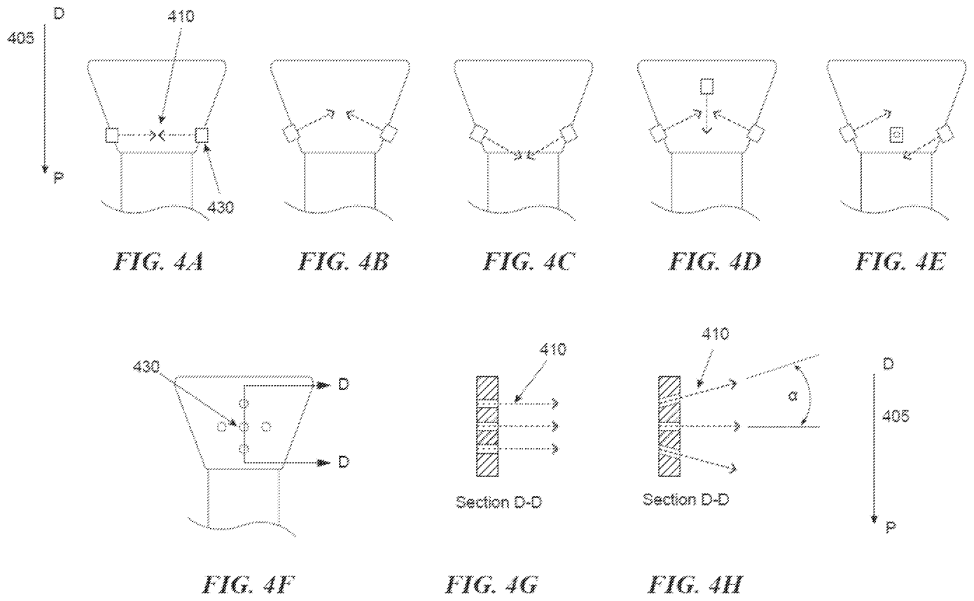

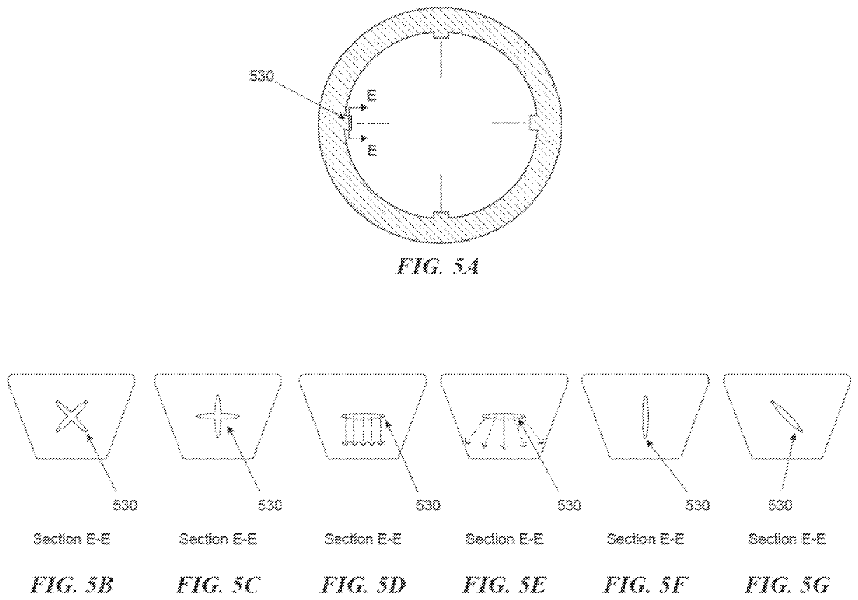

Referring to FIGS. 3A-3H, ports 330 can be arranged along various axial positions of the thrombus removal system. The thrombus removal system can include a flow axis 305 that is aligned with a general direction (e.g., distal-to-proximal) of flow for fluid that is aspirated therein. In some embodiments, a position of a port 330 comprises a) near a base of, b) in a middle portion of, c) in a distal portion of, or d) proximal to, a funnel portion 320 of the thrombus removal system. Although the embodiments illustrated herein show only a single port 330, it should be understood that any of these embodiments can include multiple ports arranged around the funnel or proximal to the funnel. For example, any of the port arrangements illustrated in FIGS. 2A-2E can be implemented a) near a base of, b) in a middle portion of, c) in a distal portion of, or d) proximal to, a funnel portion 320 of the thrombus removal system.

In some embodiments, at least two ports 330 are aligned along flow axis 305 (e.g., FIG. 3E). In some embodiments, at least two ports 330 are arranged at a different axial position along the flow axis 305 (e.g., FIGS. 3F, 3G, 3H). In some embodiments, at least two ports 330 are arranged (e.g., along a perimeter of the thrombus removal system) along a given axial position of the flow axis 305. As with above, any of the port arrangements illustrated in FIGS. 2A-2E can be implemented into the port arrangements illustrated in the embodiments of FIGS. 3F, 3G, and 3H.

FIGS. 4A-4H depict various configurations of fluid streams 410 that are directed from respective ports 430. A fluid stream 410 can be directed along a path that is substantially orthogonal (e.g., FIG. 4A), proximal (e.g., FIG. 4C), and/or distal (e.g., FIG. 4B) to the flow axis 405 (which is like to flow axis 305). In some embodiments, at least two fluid streams are directed in different directions with respect to the flow axis 405 (e.g., FIG. 4E). In some embodiments, at least one fluid stream is directed in a same direction (e.g., distally) with respect to the flow axis 405 and at least another fluid stream is directed in a different direction (e.g., orthogonally or proximally, as shown in FIG. 4D). In some embodiments, at least a first fluid stream is directed substantially orthogonally to the flow axis, at least a second fluid stream is directed proximally, and at least a third fluid stream is directed distally with respect to the flow axis 405. An angle α may characterize an angle α fluid stream 410 is directed with respect to an axis that is orthogonal to the flow axis 405 (e.g., as shown in section D-D of FIGS. 4G and 4H). An intersection region of fluid streams can be within an interior portion of the thrombus removal system, and/or exterior (e.g., distal) to the thrombus removal system. In some embodiments, a fluid stream that is directed by a port 430 in a nominal direction (e.g., distally) is deflected along an altered path (e.g., proximally) by (e.g., suction) pressure generated by the aspiration mechanism during operation.

FIGS. 5A-5G illustrate a variety of exit aperture geometries with which ports 530 can be configured in accordance with embodiments of the present technology. Aperture geometries can comprise an oval, circular, cross (“x” shape), “t” shape, rectangle, or square shape. A fluid stream that is delivered from the port 530 can comprise substantially laminar flow (e.g., at the aperture), or a turbulent flow (e.g., that fans or outward). For example, FIG. 5B shows a “x” shaped exit aperture geometry. FIG. 5C shows a “t” shaped exit aperture geometry. FIGS. 5D-5G show variations of an oval shaped exit aperture geometry. As shown in FIG. 5D, the fluid stream can be substantially laminar flow, and in FIG. 5E, the flow can fan outward.

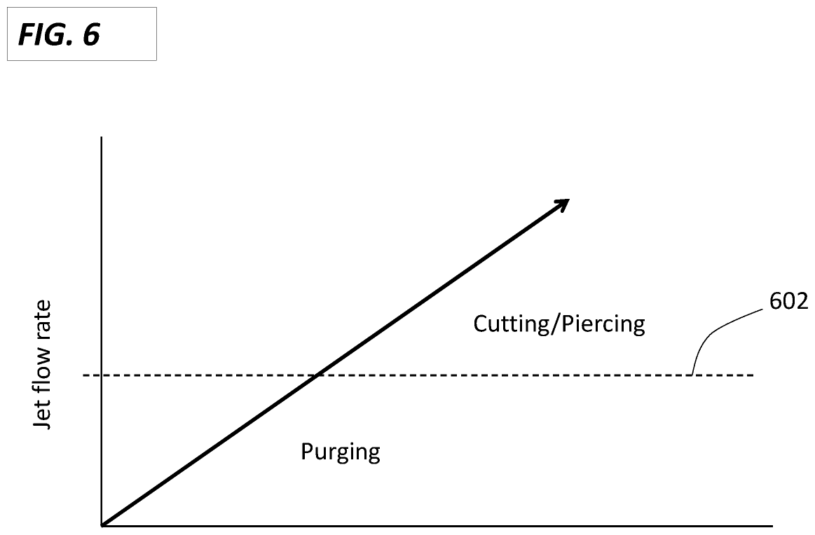

Referring to FIG. 6, a chart is provided that illustrates the relationship between the exit velocity or flow rate (avg.) of the jet(s) and the mechanism of action with one or more thrombi engaged with the thrombus removal device (i.e., engaged in the funnel or with the aspiration lumen). Generally, at lower jet flow rates (e.g., below 10 m/s depending on different parameters like the formulation of the clot and the jet configuration), the jets serve to assist with purging of the thrombus or thrombi into the aspiration lumen (especially when the funnel is occluded or partially occluded by the clot). This purging can include the function of pushing the thrombus into and or through the aspiration lumen and also providing fluid into the funnel and into the aspiration lumen to assist with clot removal. The purging may also assist with breaking up soft, loose material on the surface of the clot but it will not be able to break through harder material. However, once the jet flow rate begins to exceed a cutting threshold 602, the jets begin to cut the thrombus or thrombi surface to break the thrombus into small fragments which can then be more easily aspirated into the aspiration lumen of the thrombus removal device. It has also been found that at sufficiently high velocity the jet(s) will pierce the clot surface and penetrate through to the inner part of the clot. In some embodiments, the threshold comprises a jet flow rate that ranges from 10-12 m/s. In other embodiments, an ideal cutting or piercing flow rate of the jets range from 10-15 m/s, or alternatively, from 12-15 m/s.

The above detailed description of embodiments of the technology are not intended to be exhaustive or to limit the technology to the precise forms disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Unless the context clearly requires otherwise, throughout the description and the examples, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. As used herein, the phrase “and/or” as in “A and/or B” refers to A alone, B alone, and A and B. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

1. A thrombus removal system, comprising:
an elongated catheter device including:
a distal portion configured to be positioned within a blood vessel of a patient, the distal portion comprising an inner wall forming an aspiration lumen, and an outer wall, and a fluid lumen formed in a space between the inner wall and the outer wall;
a manifold formed near a distal end of the fluid lumen, the manifold including at least two fluid ports formed therein and adapted for fluid communication with the fluid lumen to direct respective fluid streams from the at least two fluid ports;
a proximal portion configured to be positioned external to the patient, the aspiration lumen extending from the distal portion to the proximal portion;
an aspiration mechanism positioned external to the patient and fluidly coupled with the aspiration lumen, the aspiration mechanism configured to reduce a pressure at the distal portion (a) to engage a thrombus therewith and/or (b) to draw the thrombus and/or thrombus fragments proximally; and
a fluid delivery mechanism configured to supply fluid through the fluid lumen.

2. The system of claim 1 wherein the inner wall and the outer wall are concentrically arranged such that the fluid lumen is generally annular in cross-section.

3. The system of claim 1, further comprising at least one fluid wall disposed in the space between the inner and outer walls, the at least one fluid wall forming the fluid lumen.

4. The system of claim 1, wherein the manifold is formed from the inner wall and the outer wall.

5. The system of claim 1, wherein the at least two fluid ports are formed within the inner wall of the manifold.

6. The system of claim 5, wherein the inner wall has a first thickness in a first region within the manifold, and a second thickness proximal to the first region.

7. The system of claim 6, wherein the first thickness is greater than the second thickness.

8. The system of claim 7, wherein the first thickness is about twice that of the second thickness.

9. The system of claim 6, wherein the first thickness ranges from 0.10 mm to 0.60 mm and the second thickness ranges from 0.20 mm to 0.70 mm.

10. The system of claim 6, wherein the first thickness is selected to provide a generally laminar flow for the respective fluid streams.

11. The system of claim 1, wherein a cross-sectional dimension of the two or more ports varies along its length.

12. The system of claim 11, wherein the two or more ports are conical along their lengths.

13. The system of claim 12, wherein a smallest dimension of the two or more ports is positioned at a distal end of the two or more ports.

14. The system of claim 1, wherein the respective fluid streams are configured to intersect near at least one intersection region.

15. The system of claim 14, wherein at least one intersection region is located proximally with respect to at least one of the at least two fluid ports.

16. The system of claim 14, wherein at least one intersection region is located distally with respect to at least one of the at least two fluid ports.

17. The system of claim 1, wherein the manifold is configured to increase a flow rate of the fluid in the fluid lumen.

18. The system of claim 1, wherein the manifold is configured to increase a pressure of the fluid in the fluid lumen.

19. The system of claim 1, further comprising a funnel positioned at a distal end of the distal portion, the funnel being configured to engage with the thrombus.

20. The system of claim 19, wherein the manifold is disposed proximally of the funnel.

21. The system of claim 19, wherein the manifold is integrated into the funnel.

22-35. (canceled)
